(19) United States
(12) Patent Application Publication
(10) Pub. No.: US 2016/0021939 A1
(43) Pub. Date: Jan. 28, 2016

(54) Bandeau For Hands-Free Breast Pumping

(71) Applicant: HONEYSUCKLE PRODUCTS, LLC, Marietta, GA (US)

(72) Inventor: Nossi Taheri, Atlanta, GA (US)

(73) Assignee: HONEYSUCKLE PRODUCTS, LLC, Marietta, GA (US)

(21) Appl. No.: 14/339,419

(22) Filed: Jul. 23, 2014

(51) Int. Cl. A41C 3/04; A61M 1/06

(52) U.S. Cl. CPC A41C 3/04; A61M 1/06; A61M 2209/088

(57) ABSTRACT

An apparatus for facilitating hands-free breast pumping includes a bandeau that extends around a woman's chest, the bandeau having a pair of openings each at least partially occluded by a pair of diaphragm each for receiving a component of a breast pump, such a breast cup and nozzle. Each diaphragm is defined by a plurality of panes arranged in an overlapping formation, such that each pane is secured along the perimeter of the diaphragm and includes an opposing free edge, the diaphragm includes a central variable aperture that is defined at the intersection of at least two, and possibly all of the free edges of the panes. Each pane overlaps one other pane and is overlapped by another pane so that the aperture is accessible by manipulating the panes to insert the pump components.

### TECHNICAL FIELD

The technical field relates generally to a garment that facilitates use of a breast pump apparatus, and more specifically, to a bandeau that positions and holds elements of breast pumping device in place for hands-free pumping or to free the woman's hands for manual pumping.

### BACKGROUND

With the advent of breast pumping, a mother can reap the benefits of providing optimal nutrition to her infant, while maintaining the convenient option to bottle-feed at least on occasion.

Pumping can be accomplished using manual hand pumps, or with electric or battery-powered pumps—in either case, the process takes time. For example, a mother may pump each breast for 10-30 minutes per session. The cup (sometimes referred to as a shield or flange) that engages each breast must be held in close contact to maintain adequate suction.

Ideally, the mother will not have to hold the cup in place, nor does the entire breast shield assembly hang uncomfortably on the mother's breast as its receptacle fills with milk. Otherwise, the mother's hands are not free for other activities, such as stimulating let-down, manual pumping, or for occupying the pumping time with other activities, such as reading or using a computer.

The breast shield assembly is also ungainly in that it will not fit inside of typical top clothes or undergarments, so without a specialized garment, a mother would sacrifice modesty during pumping. This is especially problematic when the mother must pump away from home.

Such specialized garments exist, attempting to serve the dual purposes of coverage and supporting the breast shield assembly in optimal relation to the breast. However, such garments fall short in fit, comfort and adjustability. For example, a heavy reliance on snaps, zippers or hooks to securely position the cup or to provide adjustability creates discomfort in the sensitive breast area. A need exists in the industry to address the aforementioned deficiencies and inadequacies.

### SUMMARY

The various embodiments of the present disclosure overcome the shortcomings of the prior art by providing a wearable apparatus for use during breast pumping, the apparatus including a bandeau that extends around a woman's chest. The bandeau has a pair of openings that correspond to the position of the woman's breasts, and a diaphragm covering all or a portion of each opening in the bandeau. The diaphragm is for receiving and securely retaining a breast cup at least in part so that the woman's hands are free for other activities during the pumping process.

Each diaphragm is similar to a camera lens, in that it includes a plurality of panes, each pane having at least one edge that is secured along the perimeter of the opening as well as at least one free edge. The panes overlap one another, in that each free edge of one pane overlies at least a portion of another pane and underlies at least a portion of yet another pane. The free edge of each pane is also disposed at an angle with respect to the free edge of each adjacent pane and, in some embodiments, the free edge of each pane is substantially parallel to the free edge of an opposing pane. In certain embodiments, the overlapping panes have a common intersection point, which may be at the center of the opening, and which defines a variable aperture in the diaphragm. The aperture may be completely occluded by the overlapping panes, which are formed from a flexible sheet material such as stretchable fabric. As such, the aperture can be accessed by manipulating the panes at the intersection point. In other embodiments, the perimeter of the variable aperture is defined by a portion of each of the free edges of the overlapping panes, such that the variable aperture is not occluded but is instead bounded by the panes, and the extent of the aperture can be increased by manipulating the panes.

In some embodiments, the bandeau is fully adjustable to conform to the size and shape of the wearer. The primary components of the bandeau are a pair of panels, left and right, that are adjustably connectable at opposing ends by either or a combination of infinitely adjustable connectors and incrementally adjustable connectors.

In certain embodiments, a restrictive connection connects each pair of adjacent panes to one another to reinforce and control the variability of the aperture.

In certain embodiments, the diaphragm is removable, replaceable, and rotatable. For instance, the diaphragm may consist of a separable assembly of panes that are arranged in an overlapping and angular relation and secured to one another. Then, the assembly can be removably affixed to the bandeau and over the opening via a decouplable connector, such as a line of snap buttons encircling the perimeter of the diaphragm and that align with receiving snap components on the bandeau, encircling the opening. In this fashion, the user can wash diaphragms separately, or replace them altogether to maintain optimal cleanliness without over-washing the bandeau itself, and the user can rotate the diaphragm as desired. As such, the diaphragm may be formed from a different material than the bandeau, to optimize cost or performance. In fact, the diaphragm may be formed from a material that has different characteristics or properties, such as cost, elasticity, strength, opacity, thickness, weight, color, permeability, texture and the like.

The foregoing has broadly outlined some of the aspects and features of the various embodiments, which should be construed to be merely illustrative of various potential applications of the disclosure. Other beneficial results can be obtained by applying the disclosed information in a different manner or by combining various aspects of the disclosed embodiments. Accordingly, other aspects and a more comprehensive understanding may be obtained by referring to the detailed description of the exemplary embodiments taken in conjunction with the accompanying drawings, in addition to the scope defined by the claims.

### DESCRIPTION OF THE DRAWINGS

FIG. 1 is a front view of a bandeau for hands free breast pumping according to an exemplary embodiment.

FIG. 2 is a front view of a front closure according to the exemplary embodiment of FIG. 1.

FIG. 3 is another front view of a front closure of FIG. 1.

FIG. 4 is a rear view of the back closure of the exemplary embodiment of FIG. 1.

FIG. 5 is a front view of the opening of the exemplary embodiment of FIG. 1.

FIG. 6 is a rear view of the opening of the exemplary embodiment of FIG.

FIG. 7 is a front view of an opening according to an alternate embodiment.

FIG. 8 is an exploded view of a portion of an alternative bandeau with a replaceable diaphragm.

### DETAILED DESCRIPTION

As required, detailed embodiments are disclosed herein. It must be understood that the disclosed embodiments are merely exemplary depictions of various and alternative forms. As used herein, the word “exemplary” is used expansively to refer to embodiments that serve as illustrations, specimens, models, or patterns. The figures are not necessarily to scale and some features may be exaggerated or minimized to show details of particular components. In other instances, well-known components, systems, materials, or methods that are know to those having ordinary skill in the art have not been described in detail in order to avoid obscuring the present disclosure. Therefore, specific structural and functional details disclosed herein are not to be interpreted as limiting, but merely as a basis for the claims and as a representative basis for teaching one skilled in the art.

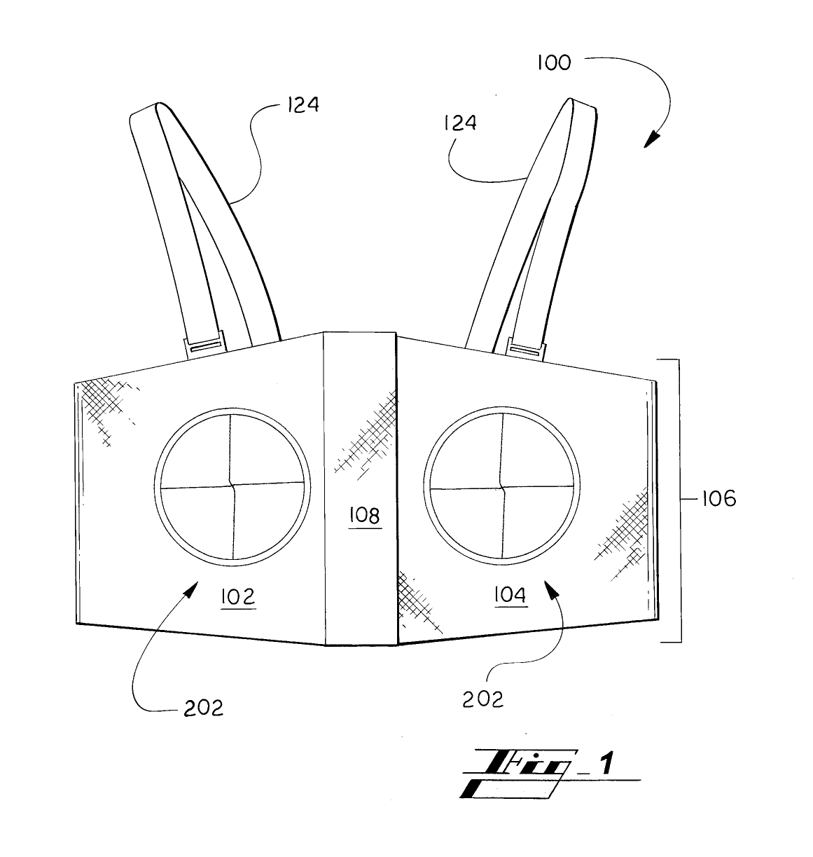

Referring to FIG. 1, an exemplary bandeau 100 includes two panels 102, 104 that can couple together along complementary ends to form a chest band 106 for encircling a woman's chest. More specifically, a right panel 102 and a left panel 104, corresponding to the right and left sides of a woman's body, include opposing divergent longitudinal top and bottom edges and opposing substantially parallel lateral end edges. Each panel 102, 104 has a substantially trapezoidal shape, although edges and corners may be curved or rounded and are not necessarily continuous, and the panels may take other shapes and configurations.

### Adjustability

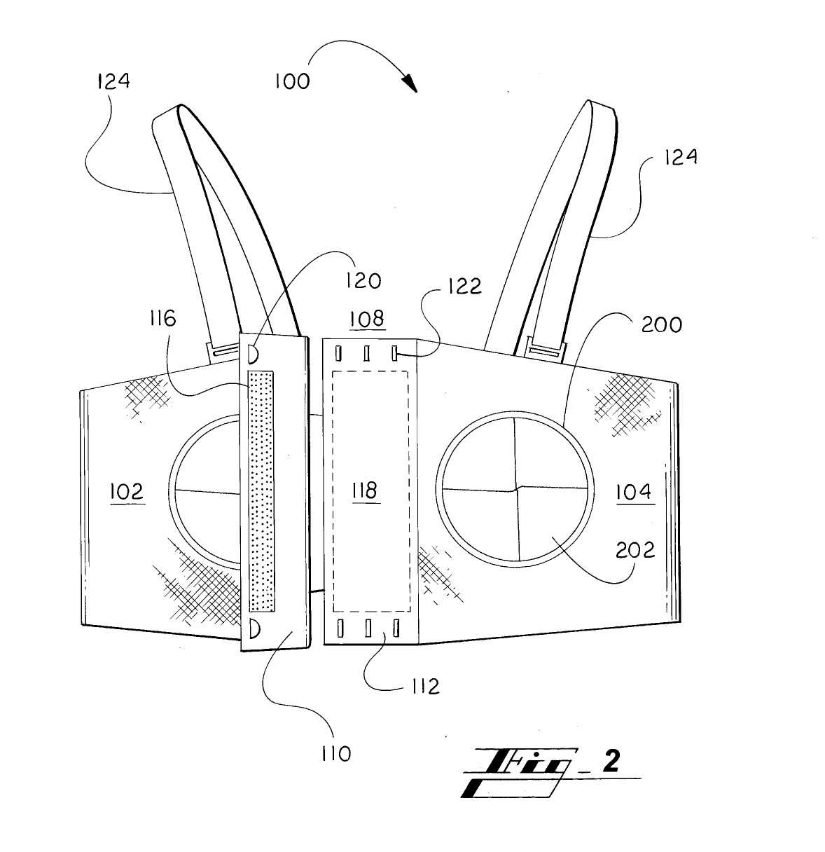

Referring now to FIGS. 1 and 2, the bandeau 100 includes a front closure 108 for releasably and preferably adjustably attaching a right front flap 110 that extends along an end edge of the right panel 102 to a left front flap 112 extending from and end edge of the left panel 104. It should be noted that the flaps 110, 112 may be continuous and integral to the respective panels 102, 104.

In this embodiment, as best seen in FIG. 4, the right panel 102 also includes back closure 114 for releasably and preferably adjustably attaching the right panel 102 to the left panel 104.

As stated above, both the front closure 108 and the back closure 114 can releasably and preferably adjustably connect the left panel 102 and the right panel 104 together, which thereby assembles the bandeau 100. The bandeau 100 can extend around a woman's chest and is sizable to conform to a variety of sizes and shapes, particularly to fit different women or to adjust to fluctuations in weight or breast fullness of the same woman. In the example shown, this is accomplished without sacrificing comfort using a combination of infinitely adjustable connectors employing hook and loop material such as VELCRO, and incrementally adjustable connectors employing hook and bar linkages.

With reference to FIG. 2, for example, the front closure 108 may include one or more first connectors 116 formed on the right front flap 110, each first connector 116 being configured to attach the right front flap 110 onto any portion of the left front flap 112, thereby providing infinite adjustability. In the exemplary embodiment, a single first front connector 116 is provided as a VELCRO strip that is sewn onto the inside surface of the right front panel 102. The first front connector 116 may alternatively include a number of such strips and the like. The left front flap 112 includes a front attachment zone 118 that is configured to receive the first front connector 116 at any point on the front attachment zone 118. The first front connectors 116 couple with the attachment zone 118 to form the front closure 108. As used herein, the term “attachment zone” refers to an area which receives the first front connector 116 at virtually any point on the attachment zone, thereby permitting infinite adjustment.

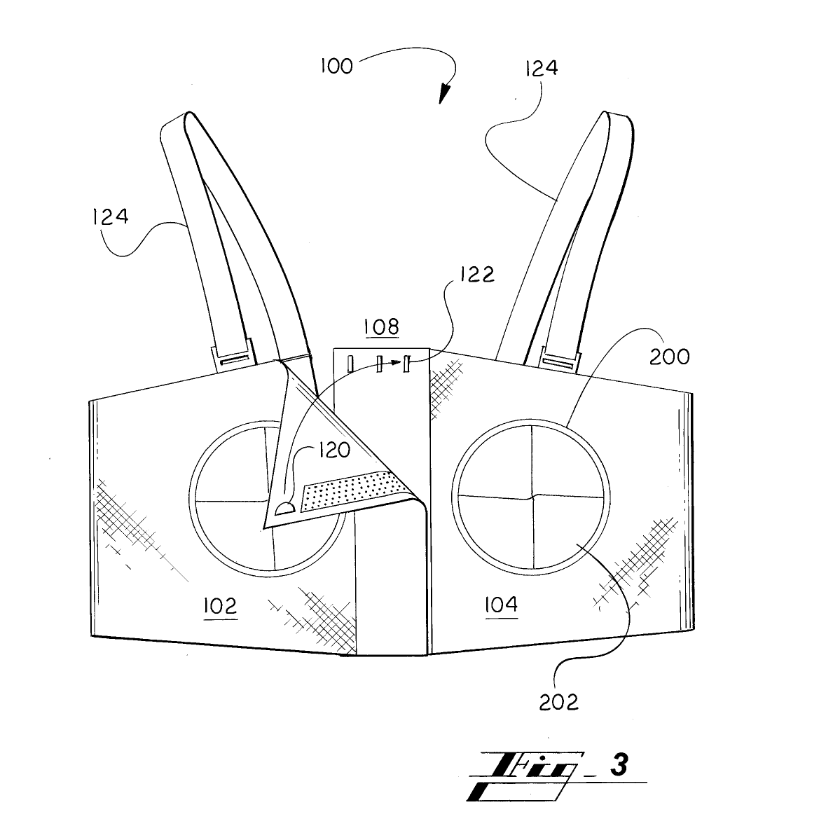

As alternative means for receiving the first front connectors 116, the front closure 108 may also or additionally include one or more second front connectors 120, such as the pair of hooks disposed at the top end edge and at the bottom end edge of the right front panel 102. The second front connectors 120 couple with receiving elements 122, such as the incrementally spaced bars shown in FIG. 3, to form or reinforce the front closure 108.

Referring again to FIG. 4, the back closure 114 may also include a combination of infinitely adjustable connectors and incrementally adjustable connectors, or as in the embodiment shown, only an infinitely adjustable connector arrangement may be used. Specifically, the exemplary embodiment includes at least one rear connector 119, such as a VELCRO strip that is sewn onto the inside surface of the right front panel 102. The rear connector 119 can be secured at any point along a rear attachment zone 121. In the example shown, the rear attachment zone 121 comprises the entire rear portion of the left panel 104, which is constructed from a material with loops that engage the VELCRO strip that defines the exemplary rear connector 119.

These specific closures are merely exemplary, as various alternatives are available to function as means for infinite adjustability, such as magnets or magnetic strips, reusable adhesive strips such as tacky strips or rubberized tape and the like. Similarly, numerous alternatives that function as means for incremental adjustability include sequentially spaced sets of fasteners, such as G-hooks, bra clickers, garter clips, magnetic clips, slide locks, hooks and eyes, snaps, buttons, buckles, clasps, ties, and the like. In some scenarios, it is possible to provide one male second connector 120 on one of the left panel 102 or the right panel 104 and multiple female components sequentially spaced on the other of the left panel 102 or the right panel 104, or vice versa. Alternatively, it is possible to provide a single female receiving element 122 that couples with multiple male second connectors 120.

To further increase the utility and adjustability of the bandeau 100, a pair of optional and preferably moveable and removeable straps 124 may be provided and utilized by a user. The length and position of straps 124 can also be adjusted to further increase utility and comfort.

### Secure Retaining Diaphragms

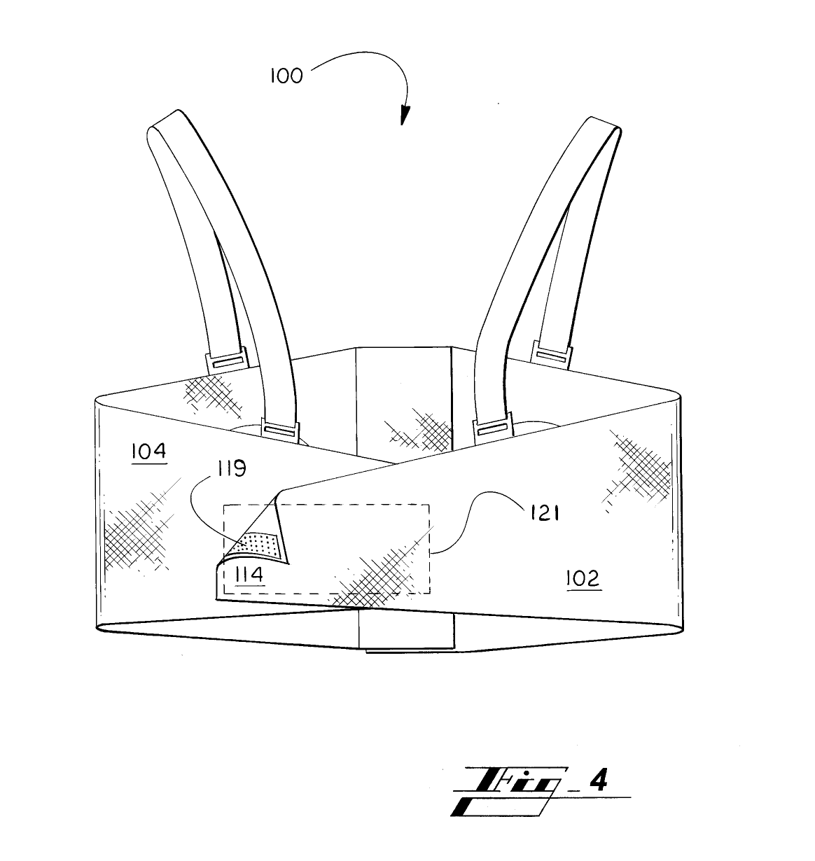

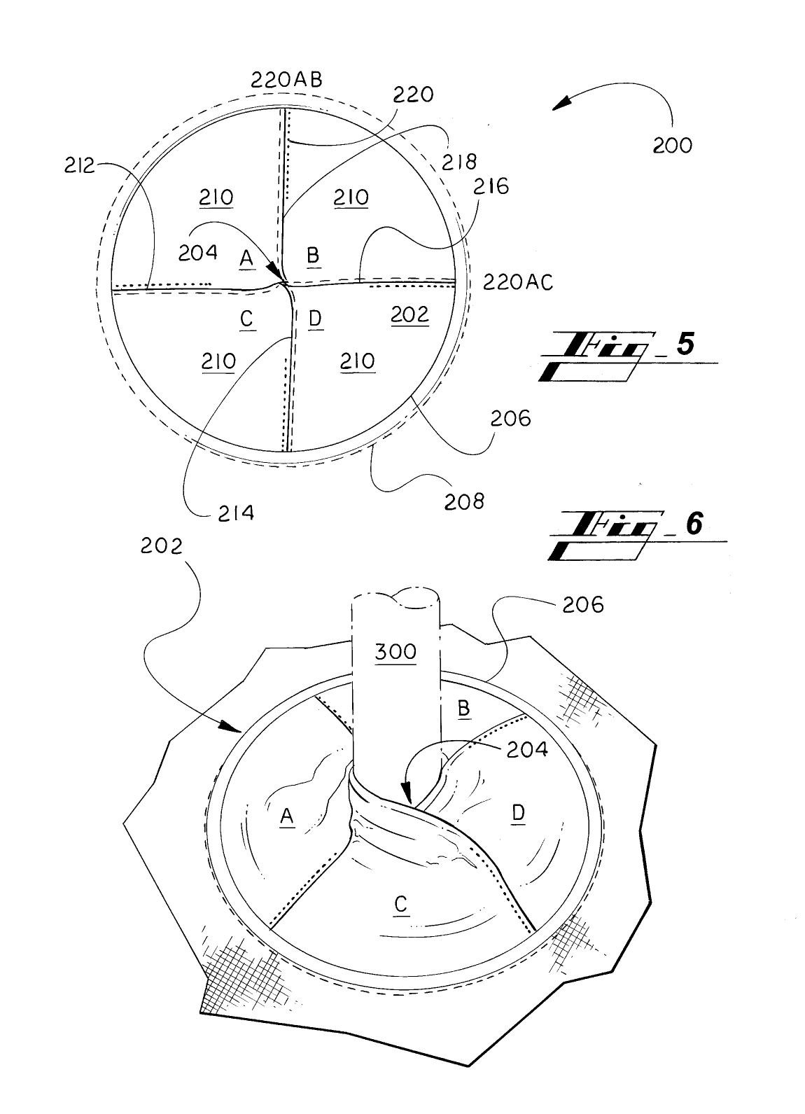

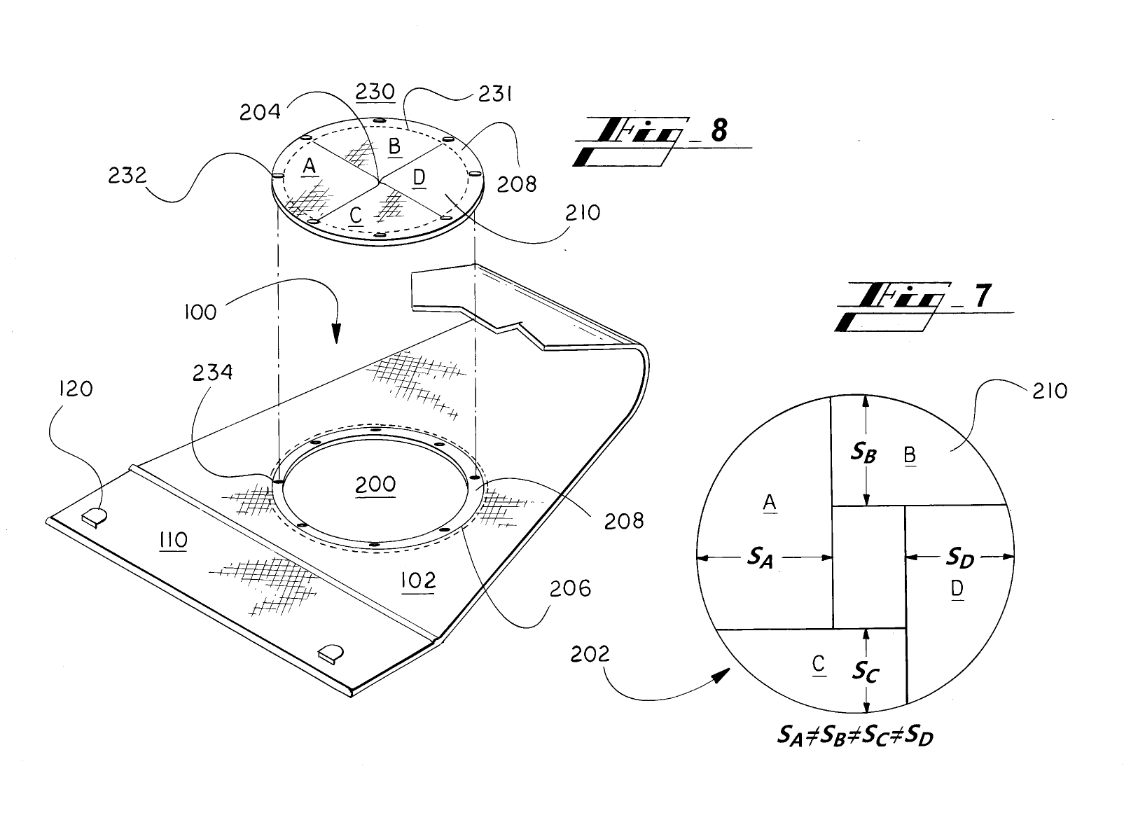

Referring particularly to FIGS. 5 and 6, the bandeau 100 includes two openings 200, one for each breast. Each opening 200 is accessible via a diaphragm 202 that defines a variable aperture 204 for receiving and for retaining breast pump components. To further illustrate the environment of the disclosure, as opposed to for limitation, a nozzle 300 that extends through the aperture 204 from a cup (not shown) disposed between the breast (not shown) and the diaphragm 202 is shown in FIG. 6. The exemplary opening 200 is circular to conform to the shape of a typical breast, although it can have any suitable shape, including oval (i.e., egg-shaped), elliptical, or polygonal (assuming a shape such as a triangle, square, rectangle, or octagon). The diaphragm 202 is connected along the perimeter 206 of the opening 200, which in the exemplary embodiment is defined by the circumference of the opening 200. A line of connection 208, such as the running stitch used in the exemplary embodiment, attaches the diaphragm 202 along the perimeter 206. As used herein, any line of connection may consist of several cooperating lines or points of connection that may be distributed or substantially continuous.

Referring now to FIG. 5, the diaphragm 202 is formed at least in part from a number of panes 210, each pane 210 being secured along at least one of its edges to the perimeter 206 of the opening 200, and each pane 210 partially overlapping at least one other pane 210 and being partially overlapped by at least one other pane 210. In this manner, the panes 210 of the diaphragm 202 cooperate such that the opening 200 is substantially covered by overlapping panes 210, and the free edges of the panes 210 share a common point of intersection P, preferably defining the center of the opening, at which common intersection point P the variable aperture 204 is defined.

In the exemplary embodiment, there are four panes 210, which are distinguished one from the other by the letters A, B, C and D. Each pane 210 is substantially semicircular, and is stitched or otherwise attached along its arcuate edge to the opening 200, with its straight edge spanning the opening 200, thereby bisecting the opening 200 in certain embodiments. Each pane 210 is disposed at a right angle with respect to each adjacent pane 210, and is at a 180 degree orientation with respect to an opposing pane 210, although the angular relations may vary with the number of panes, or as desired. In the embodiment shown, for example, pane A is secured along the top of the opening 200 (centered at 0 degrees), adjacent pane B is secured along the right side of the opening 200 (centered at 90 degrees), opposing pane C is secured along the bottom of the opening 200 (centered at 180 degrees), and pane D is secured along the left half of the opening 200 (centered at 270 degrees). The free edge 212 of pane A extends under adjacent pane B, overlies pane C and is perpendicular to the free edge 214 of pane C. The free edge 212 of pane A is also parallel to the free edge 216 of pane D. The same relative positioning applies to each of the panes A, B, C, D. The position of the pane arrangement can be rotated incrementally as desired while maintaining the angular relation of one pane 210 to the next pane 210, and the angular relationship between panes is not necessary consistent.

In certain embodiments, an optional restrictive connection 220 is also employed to restrict the ability of the panes 210 to move relative one another, thereby reinforcing the aperture 204 while still facilitating manual manipulation of the aperture to insert breast pump components. More specifically, for example, pane A may be partially connected to pane B along restrictive connection 220AB, and pane A may also be partially connected to pane C along restrictive connection 220AC. Similar restrictive connections 220 are provided to secure pane B to pane D and to secure pane C to pane D. In the exemplary embodiment, the restrictive connections 220 extend radially from the perimeter 206 toward the common intersection point C. The extent to which the restrictive connections 220 approach the common intersection point C controls the maximum extent to which the variable aperture 204 can be opened. In the examples shown, the restrictive connection 220 is a line of stitches, although other devices may be used, such as spaced tack stitches, iron-on fabric tape, and the like.

Although four panes 210 are present in the exemplary embodiment, the number of panes 210 is a design choice, as it is contemplated that alternative configurations can be selected, and that different characteristics of the application may drive the choice. In general, increasing the number of panes 210 creates a tighter variable aperture 204, and provides additional breast coverage. As described above, each pane 210 both overlaps and is overlapped by at least one other pane 210, which can be accomplished with as few as three panes 210.

The size of one or more of the panes 210 is also variable. Referring to FIG. 5, each pane 210 shown is semicircular, and as such, covers half of the opening 200, which is circular in the exemplary embodiment. Where the opening 200 is circular, the panes 210 are effectively arcuate. However, referring now to FIG. 8, rather than bisecting the opening 200, a pane 210 may be defined by a chord defining an arc that has a smaller sagitta S than the radius R (i.e., covering less than half of the diameter of the opening 200), and thus an arcuate pane 210 is not necessarily semicircular. The sagitta of the panes 210 can be adjusted to control the extent to which the variable aperture 204 can be occluded by the panes 210 as well as the degree to which the variable aperture 204 can be opened. In FIG. 8, the sagitta S_{A} of pane A is less than R (one half of the diameter of the opening 200), as are the sagitta S_{B}, S_{C}, and S_{D} of the panes B, C and D, and the sagitta S_{A}, S_{B}, S_{C}, and S_{D} are not necessarily equal to one another. As a result, the aperture 204 is not occluded such that it is open to some extent regardless of whether the panes 210 have been manipulated or whether the aperture 204 has any pump component 300 extending through it. By varying the sagitta S_{A}, S_{B}, S_{C}, and S_{D}, aperture 204 is not necessarily centered in the opening 200, which may account for natural asymmetry of breasts, which may hang to one side or the other, or may otherwise optimize positioning of a pump component 300.

The shape of each pane 210 is also variable, without departing from the scope of the disclosure. For example the free edge 212, 214, 216, 218 of a pane 210 need not be straight, as a curved or angled edge would not depart from the principles of the invention. It follows that the opposing secured edge of each pane 210, which is secured along line of connection 208 can have any suitable shape. Furthermore, where the opening 200 is not circular, a pane 210 may assume any suitable complementary shape.

The bandeau 100 is constructed from soft, pliable material, such as a soft fabric. Where hook and loop closures are utilized, any or all of the material is preferably capable of serving as the loop bearing surface.

### Replaceable and Rotatable Diaphragm

Referring to FIG. 8, an alternative embodiment of bandeau 100 comprises a removable, replaceable, and rotatable diaphragm 230. This embodiment introduces additional benefits and advantages. For example, the diaphragm 230 maintains the structural advantages of the fixed diaphragm 202, but allows the woman to employ a single bandeau 100 with multiple diaphragms 230, which can be washed between uses to extend the life of the entire apparatus. In fact, the bandeau 100 is spared frequent washing, while clean replacement diaphragms 230 can be kept in contact with the breast (not shown) and the pump components 300. The diaphragm 230 can be affixed via connectors that can be decoupled for easy removal of the diaphragm 230, and that further allow affixation of the diaphragm 230 at any orientation, such that the woman can adjust the rotation of the diaphragm 230 to customize the manner in which the panes 210 are manipulated to access opening 200 via variable aperture 204.

In the example shown in FIG. 8, the panels A, B, C and D of diaphragm 230 are further secured to one another along line of connection 231, which extends adjacent to the periphery of the diaphragm 230. The diaphragm 230 includes coupling connectors 232, such as the female snap button shown, which are configured to be releasably received by an opposing coupling connector 234, such as the male snap button shown, which is arranged adjacent the perimeter 206 of the opening 200. When the diaphragm 230 is attached to the bandeau 100, the line of connection 231 may align with the perimeter 206 of the opening 200, and the diaphragm 202 overlaps the panel 102 to the line of connection 208.

### Manner of Use

As a non-limiting example, to use the bandeau 100, a woman simply connects the back closure 114, and places the straps 124 over each shoulder, with the bandeau 100 open to the front. She may then use either or both of the incrementally adjustable connectors 120 and the infinitely adjustable connectors 116 to achieve a snug and comfortable fit. Referring now to FIG. 6, the aperture 204 is accessed by spreading the panes 210 apart with two or three fingers or with the breast pump cup itself. The overlapping panes 210 spread apart as needed to accommodate wider elements of the breast pump cup, such as a flared opening, while the restrictive connections 220 reinforce the frictional hold of one pane 210 on another 210 to increase the retention strength of the diaphragm 202.

The above-described embodiments are merely exemplary illustrations of implementations that are set forth for a clear understanding of principles. Variations, modifications, and combinations may be made to the above-described embodiments may be made without departing from the scope of the claims. All such variations, modifications, and combinations are included herein by the scope of this disclosure and the following claims.

What is claimed is:

1. An apparatus, comprising:
a bandeau;
an opening formed in the bandeau;
a plurality of panes that define a diaphragm that at least partially covers the opening, each pane comprising a free edge, each free edge of one pane overlying at least a portion of another pane and underlying at least a portion of yet another pane to define an overlapping arrangement;
means for affixing the diaphragm to the bandeau along a line of connection;
an aperture disposed in the diaphragm, the aperture being defined at least in part by the free edges of the plurality of panes.

2. The apparatus of claim 1, wherein the aperture being defined at least in part by the free edges of the plurality of panes comprises the aperture being defined by an intersection of at least two of the free edges of the plurality of panes.

3. The apparatus of claim 1, wherein the aperture being defined at least in part by the free edges of the plurality of panes comprises the aperture being defined by an intersection of at least two of the free edges of the plurality of panes.

4. The apparatus of claim 1, wherein the free edge of each pane is disposed at an angle with respect to the free edge of each adjacent pane.

5. The apparatus of claim 1, wherein the free edge of each pane is substantially parallel to the free edge of an opposing pane.

6. The apparatus of claim 1 wherein the diaphragm is defined in part by a line of connection that secures each pane in an overlapping arrangement.

7. The apparatus of claim 1, wherein the aperture is configured to receive and retain breast pump components without continual manual support.

8. The apparatus of claim 1, wherein the opening is substantially circular and each of the plurality of panes is substantially arcuate.

9. The apparatus of claim 6, wherein the diaphragm is removeable from the bandeau along the line of connection.

10. The apparatus of claim 9, wherein the diaphragm comprises at least one coupling connector configured to be releasably coupled to at least one opposing coupling connecter formed on the bandeau.

11. The apparatus of claim 1, wherein:
the bandeau comprises a left panel and a right panel;
the opening is a first opening in the left panel;
the bandeau further comprises a second opening in the right panel; and
the diaphragm is a first diaphragm and further comprising a second diaphragm that at least partially covers the second opening.

12. The apparatus of claim 11, further comprising:
a right front flap extending from an end of the right panel;
a left front flap extending from an end of the left panel;
a front closure for securing the left panel to the right panel along the left front flap and the right front flap, the front closure comprising:
at least one front connector associated with either the right front flap or the left front flap; and
means for receiving the at least one front connector, the means for receiving being associated with the other of the left front flap or the right front flap.

13. The apparatus of claim 12, wherein the means for receiving comprises a front attachment zone.

14. The apparatus of claim 13, wherein the at least one front connector comprises at least one hook panel, and the front attachment zone provides loops for adhesion with the at least one hook panel, thereby forming a hook and loop connection.

15. The apparatus of claim 12, wherein the means for receiving comprises at least one opposing front connector.

16. The apparatus of claim 11, further comprising:
a rear flap extending from an end of one of the left panel or the right panel;
a back closure for securing the left panel to the right panel, the back closure comprising:
at least one rear connector associated with rear flap; and
means for receiving the at least one rear connector, the means for receiving being associated with the other of the left panel or the right panel.

17. The apparatus of claim 16, wherein the means for receiving comprises an attachment zone.

18. The apparatus of claim 17, wherein the at least one rear connector comprises at least one hook panel, and the rear attachment zone provides loops for adhesion with the at least one hook panel, thereby forming a hook and loop connection.

19. The apparatus of claim 18, wherein the means for receiving comprises at least one opposing rear connector.
